(19) United States
(12) Patent Application Publication
(10) Pub. No.: US 2023/0062880 A1
(43) Pub. Date: Mar. 2, 2023

(54) ASSAYS AND METHODS FOR MISCARRIAGE RISK ASSESSMENT

(71) Applicant: GENOMIC PREDICTION, North Brunswick, NJ (US)

(72) Inventor: Laurent Christian Asker Melchior TELLIER, New Brunswick, NJ (US)

(73) Assignee: GENOMIC PREDICTION, North Brunswick, NJ (US)

(21) Appl. No.: 17/929,508

(22) Filed: Sep. 2, 2022

Related U.S. Application Data
(60) Provisional application No. 63/240,173, filed on Sep. 2, 2021.

(51) Int. Cl. C12Q 1/6827; C12Q 1/6881; G16H 50/30; G16H 50/20

(52) U.S. Cl. CPC C12Q 1/6827; C12Q 1/6881; G16H 50/30; G16H 50/20; C12Q 2600/158; C12Q 2600/124

(57) ABSTRACT

Disclosed herein are methods and systems of generating successful pregnancy probability descriptive of the embryo's potential for a successful pregnancy if implanted. The successful pregnancy probability generated may be used in methods and systems for selecting an embryo from a set of non-aneuploid embryos or for ranking a set of non-aneuploid embryos for suitability for intrauterine transfer from the set of non-aneuploid embryos.

### CROSS-REFERENCE TO RELATED APPLICATIONS

The present application claims priority to U.S. Provisional Application No. 63/240,173, entitled “ASSAYS AND METHODS FOR MISCARRIAGE RISK ASSESSMENT,” filed on Sep. 2, 2021.

### BACKGROUND OF THE INVENTION

In vitro fertilization (IVF) is the most effective treatment of infertility. Each year, clinicians perform more than 2.5 million IVF cycles globally. As clinical and laboratory methods have improved, so has the efficiency of producing blastocysts suitable for intrauterine transfer. As a result, IVF patients and physicians are often faced with determining which specific embryo to transfer to impregnate the prospective mother. The default strategy for choosing which embryo to transfer involves ranking embryos through careful microscopy-based characterization of development and morphology. However, preimplantation genetic testing (PGT) has become a routine method for embryo selection, now implemented in 40% of all in vitro fertilization (IVF) cycles in the United States. PGT is most commonly applied to select euploid (or likely euploid, hereafter described as non-aneuploid) embryos for transfer, while avoiding those embryos designated as aneuploid (PGT-A). The primary objective of PGT-A is to improve the probability of success of IVF in the first attempted embryo transfer. Again, the default strategy for choosing which non-aneuploid embryo to transfer involves ranking embryos through careful microscopy-based characterization of development and morphology.

Accordingly, there is a need for methods and assays for determining the risk of pregnancy loss following IVF embryo implantation, as well as for embryo selection and the reduction of the risk of miscarriage, based upon genotyping of the embryo, supplementing the euploidy/aneuploidy determination described above.

### SUMMARY OF THE INVENTION

Described herein are tests for a genotype in a human embryo, used to generate a score or probability descriptive of the embryo's potential for a successful pregnancy if implanted (described here as a “successful pregnancy probability”), manifested as a probability of a successful pregnancy for the implanted embryo. A successful pregnancy probability may be used, for example, to designate an embryo to be at lower-than-average risk of an unsuccessful pregnancy or of not being carried to term, or it may be used, for example, to rank order several embryos on the basis of their probability for a successful pregnancy.

In one aspect, provided herein are methods of generating a successful pregnancy probability for an embryo of a mother and a father, descriptive of the embryo's potential for a successful pregnancy if implanted; the method implemented by a computer processor executing program instructions, the method comprising the steps of: (i) providing gene sequences from a genomic DNA sample obtained from the embryo; (ii) identifying a genotype at a plurality of loci in the gene sequences; and (iii) using the processor, generating for the embryo the successful pregnancy probability, wherein the successful pregnancy probability is calculated using the genotypes identified at the plurality of loci in the gene sequences and optionally aggregating structural, karyotypal, and other factors into this probability.

In another aspect, provided herein are methods of generating a successful pregnancy probability for an embryo of a mother and a father, descriptive of the embryo's potential for a successful pregnancy if implanted; the method implemented by a computer processor executing program instructions, the method comprising the steps of: (i) providing gene sequences from a genomic DNA sample obtained from the embryo; (ii) providing gene sequences measured using a genotyping array, or sequencing, from genomic DNA samples obtained from the embryo's mother and father; (iii) using the processor, assigning a genotype based on the gene sequences present or absent in the embryo and the embryo's mother and father at a plurality of loci; and using the processor, generating for the embryo the successful pregnancy probability, wherein the successful pregnancy probability is calculated using the genotypes identified at the plurality of loci in the gene sequences and optionally aggregating structural, karyotypal, and other factors into this probability

In another aspect, provided herein are methods of treatment to reduce the risk of a woman not having a successful pregnancy with an implanted embryo and/or increasing the probability of the woman having a successful pregnancy with the embryo, the method comprising the steps of: (a) determining that the woman is at risk of an unsuccessful pregnancy with the implanted embryo based on the successful pregnancy probability generated as described herein; and (b) administering low dose heparin to the woman to increase the probability that the pregnancy will be successful and/or to reduce the risk that the pregnancy will be unsuccessful.

In another aspect, provided herein are methods of selecting an embryo for suitability for intrauterine transfer from a set of non-aneuploid embryos, the method implemented by a computer processor executing program instructions, the method comprising the steps of: (i) using the processor, generating a successful pregnancy probability as described herein for each embryo of the set of non-aneuploid embryos; (ii) including, excluding, up-prioritizing or down-prioritizing the embryo from the set of non-aneuploid embryos as suitable or unsuitable for intrauterine transfer, if the successful pregnancy probability for the embryo is better than or worse than the successful pregnancy probability for another embryo of said set of non-aneuploid embryos, or if the successful pregnancy probability falls above or below a pre-determined threshold; and (iii) repeating steps (i)-(ii) for each embryo of said set of non-aneuploid embryos.

In another aspect, provided herein are methods of ranking a set of non-aneuploid embryos for suitability for intrauterine transfer, the method implemented by a computer processor executing program instructions, the method comprising the steps of: (i) using the processor, generating a successful pregnancy probability as described herein for each embryo of the set of non-aneuploid embryos; (ii) ranking each embryo from the set of non-aneuploid embryos for suitability for intrauterine transfer based on the successful pregnancy probability of that embryo, wherein the ranking is relative to the successful pregnancy probability of another embryo of said set of non-aneuploid embryos or the ranking is relative to a pre-determined standard; and (iii) repeating steps (i)-(ii) for each embryo of said set of non-aneuploid embryos.

In another aspect, provided herein are systems for generating a successful pregnancy probability for an embryo of a mother and a father, descriptive of the embryo's potential for a successful pregnancy if implanted; the system comprising: a memory and a computer processor to: (i) receive gene sequences from a genomic DNA sample obtained from the embryo; (ii) identify a genotype at a plurality of loci in the gene sequences; and (iii) generate for the embryo the successful pregnancy probability, wherein the successful pregnancy probability is calculated using the genotypes identified at the plurality of loci in the gene sequences.

In another aspect, provided herein are systems for generating a successful pregnancy probability for an embryo of a mother and a father, descriptive in the embryo's potential for a successful pregnancy if implanted; the system comprising: a memory and a computer processor to: (i) receive gene sequences from a genomic DNA sample obtained from the embryo; (ii) receive gene sequences measured using a genotyping array from genomic DNA samples obtained from the embryo's mother and father; (iii) assign a genotype based on the gene sequences present or absent in the embryo and the embryo's mother and father at a plurality of loci; and (iv) generate for the embryo the successful pregnancy probability, wherein the successful pregnancy probability is calculated using the genotypes identified at the plurality of loci in the gene sequences.

In another aspect, provided herein are systems selecting an embryo for suitability for intrauterine transfer from a set of non-aneuploid embryos; the system comprising: a memory and a computer processor to: (i) generate a successful pregnancy probability as described herein for each embryo of the set of non-aneuploid embryos; (ii) include, exclude, up-prioritize or down-prioritize the embryo from the set of non-aneuploid embryos as suitable or unsuitable for intrauterine transfer, if the successful pregnancy probability for the embryo is better than or worse than the successful pregnancy probability for another embryo of said set of non-aneuploid embryos, or if the successful pregnancy probability falls above or below a pre-determined threshold; and (iii) repeat steps (i)-(ii) for each embryo of said set of non-aneuploid embryos.

In another aspect, provided herein are systems for ranking a set of non-aneuploid embryos for suitability for intrauterine transfer; the system comprising: a memory and a computer processor to: (i) generate a successful pregnancy probability as described herein for each embryo of the set of non-aneuploid embryos; (ii) rank each embryo from the set of non-aneuploid embryos for suitability for intrauterine transfer based on the successful pregnancy probability of that embryo, wherein the ranking is relative to the successful pregnancy probability of another embryo of said set of non-aneuploid embryos or the ranking is relative to a pre-determined standard; and (iii) repeat steps (i)-(ii) for each embryo of said set of non-aneuploid embryos.

Other features and advantages will become apparent from the following detailed description, examples, and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 schematically illustrates a system for executing one or more methods according to embodiments of the invention.

FIGS. 2A-2D depict exemplary saliva DNA sample results for gene ANXA5 M2 haplotype locus in a population of blastocysts.

FIG. 3A-3D depicts exemplary sequence data from the saliva DNA sample population of FIGS. 2A-2D. The dashed arrows indicate heterozygous presence of ANXA5 M2 haplotype.

FIGS. 4A-4D depict exemplary 7-cell sample results.

FIGS. 5A-5D depict exemplary sequence data from the 7-cell samples of FIGS. 4A-4D.

FIG. 6 depicts an exemplary PGT case.

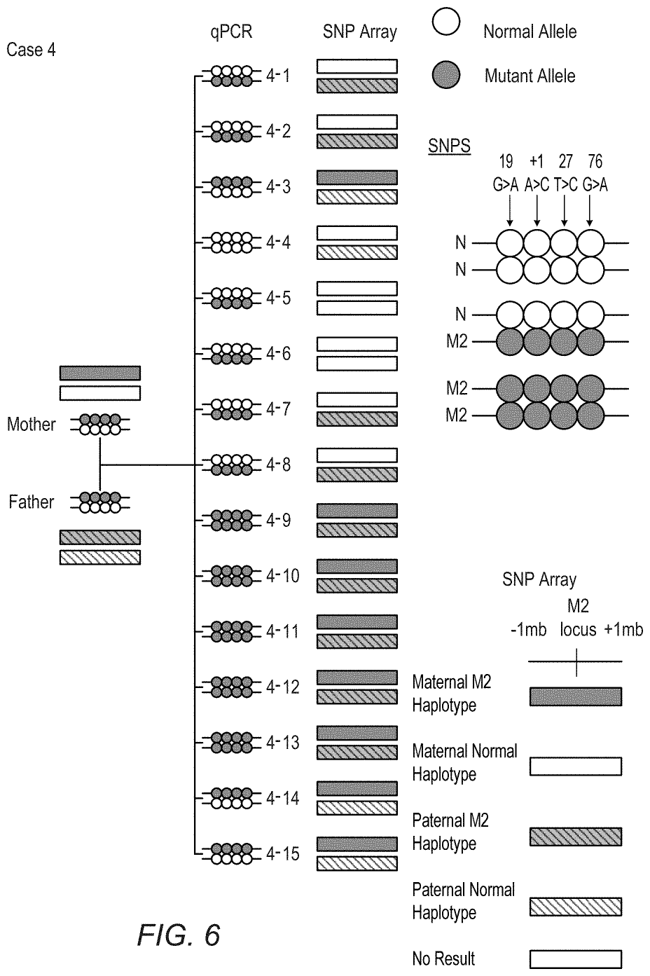

FIG. 7 schematically illustrates a display of the ranking of a set of embryos based on the successful pregnancy probability for each embryo and generated according to embodiments of the systems and methods described herein.

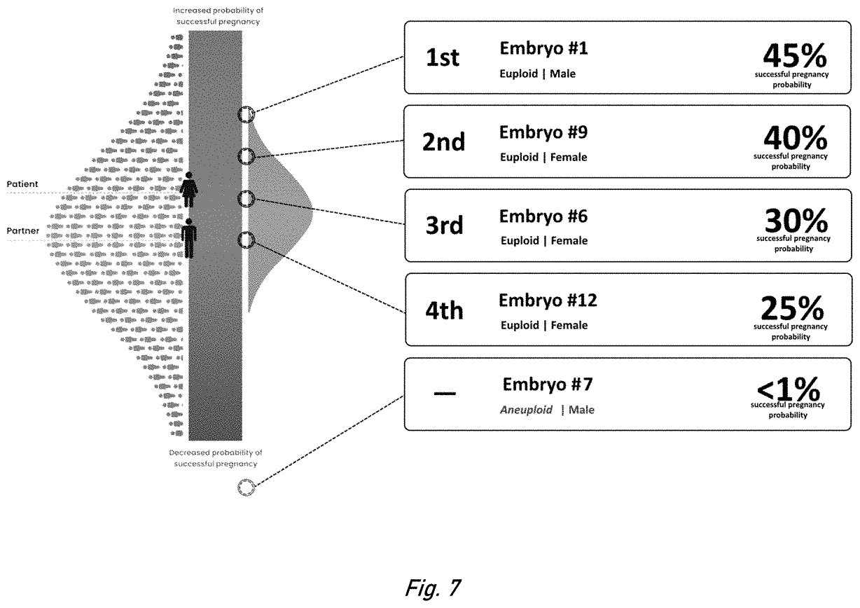

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth to provide a thorough understanding of the present invention. However, it will also be apparent to one skilled in the art that this invention may be practiced without the specific details presented herein. Furthermore, well known features may be omitted or simplified in order not to obscure the present invention.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

In accordance with embodiments of the present invention and as used herein, the following terms are defined with the following meanings, unless explicitly stated otherwise.

As used herein, “haploid cell” refers to a cell with a haploid number (n) of chromosomes. “Gametes”, as used herein, are specialized haploid cells (e.g., spermatozoa and oocytes) produced through the process of meiosis and involved in sexual reproduction. As used herein, “gametotype” refers to single genome copies with one allele of each of one or more loci in the haploid genome of a single gamete.

As used herein, an “autosome” is any chromosome exclusive of the X and Y sex chromosomes.

As used herein, “diploid cell” has a homologous pair of each of its autosomal chromosomes and has two copies (2n) of each autosomal genetic locus.

The term “chromosome”, as used herein, refers to a molecule of DNA with a sequence of base pairs that corresponds closely to a defined chromosome reference sequence of the organism in question.

As used herein, “euploid cell” or “euploid organism” refers to a cell or organism, respectively, having an exact multiple of the haploid number of chromosomes. (It may be readily understood by those skilled in the art that cells may temporarily have double the complement of chromosomes during G2 phase and M phase of the cell cycle.)

As used herein, “aneuploid cell” or “aneuploid organism” refers to a cell or organism, respectively, having a chromosome number that is not an exact multiple of the usually haploid number.

The term “gene”, as used herein, refers to a DNA sequence in a chromosome that codes for a product (either RNA or its translation product, a polypeptide) or otherwise plays a role in the expression of said product. A gene contains a DNA sequence with biological function. The biological function may be contained within the structure of the RNA product or a coding region for a polypeptide. The coding region includes a plurality of coding segments (“exons”) and intervening non-coding sequences (“introns”) between individual coding segments and non-coding regions preceding and following the first and last coding regions, respectively.

The term “gene product”, as used herein, refers to a product (either RNA or its translation product, a polypeptide) that is encoded by a gene and that has biological function.

As used herein, “locus” refers to any segment of DNA sequence defined by chromosomal coordinates in a reference genome known to the art, irrespective of biological function. A DNA locus may contain multiple genes or no genes; it may be a single base pair or millions of base pairs. As used herein, a “polymorphic locus” is a genomic locus at which two or more alleles have been identified.

As used herein, an “allele” is one of two or more existing genetic variants of a specific polymorphic genomic locus.

As used herein, a “single nucleotide polymorphism” or “SNP” is a particular base position in the genome where alternative bases are known to distinguish one individual from another. Most categories of more complex genetic variants may be reduced for analytical purposes to one or a few defining SNPs.

As used herein, a “copy number variant” or “CNV” is a deletion or duplication of a large block of genetic material that exists in a population at a frequency less than 1%. As used herein, a “copy number polymorphism” or “CNP” is a deletion or duplication of a large block of genetic material that exists in a population at a frequency of greater than 1%. Since a CNV in one population may be a CNP in a second population, the two terms may be used interchangeably.

As used herein, “genotype” refers to the diploid combination of alleles at a given genetic locus, or set of related loci, in a given cell or organism. A homozygous subject carries two copies of the same allele and a heterozygous subject carries two distinct alleles. In the simplest case of a locus with two alleles “A” and “a”, three genotypes may be formed: A/A, A/a, and a/a.

As used herein, “genotyping” refers to any experimental, computational, or observational protocol for distinguishing an individual's genotype at one or more well-defined loci.

As used herein, a “haplotype” is a unique set of alleles at separate loci that are normally grouped closely together on the same DNA molecule and are observed to be inherited as a group. A haplotype may be defined by a set of specific alleles at each defined polymorphic locus within a haploblock. As used herein, a “haploblock” refers to a genomic region that maintains genetic integrity over multiple generations and is recognized by linkage disequilibrium within a population. Haploblocks are defined empirically for a given population of individuals.

As used herein, “linkage disequilibrium” is the non-random association of alleles at two or more loci within a particular population. Linkage disequilibrium is measured as a departure from the null hypothesis of linkage equilibrium, where each allele at one locus associates randomly with each allele at a second locus in a population of individual genomes.

As used herein, a “genome” is the total genetic information carried by an individual organism or cell, represented by the complete DNA sequences of its chromosomes.

As used herein, a “genomic profile” is a representative subset of the total information contained within a genome. A genomic profile contains genotypes at a particular set of polymorphic loci.

As used herein, a genetic “trait” is a distinguishing attribute of an individual, whose expression is fully or partially influenced by an individual's genetic constitution.

As used herein, “disease” refers to a trait that is at least partially heritable and causes a reduction in the quality of life of an individual person.

As used herein, a “phenotype” includes alternative traits which may be discrete or continuous. Phenotypes may include both traits and diseases.

As used herein, “NCBI” refers to the National Center for Biotechnology Information which is a division of the National Library of Medicine at the U.S. National Institutes of Health. The NCBI operates under a Congressional mandate to develop, maintain, and distribute databases and software to the research and medical communities.

As used herein, a “variant” is a particular allele at a locus where at least two alleles have been identified.

As used herein, a “mutation” has the same meaning as a “mutant allele” which is a variant that causes a gene to function abnormally.

As used herein, a “single gene phenotype” is a phenotype that may be caused by the expression of a genotype of a single gene. As used herein, a “single gene disease” is a disease that may be caused by a mutation or mutations in a single gene.

As used herein, a “polygenic phenotype” is a phenotype that may be caused by the expression of genotypes of more than one gene. As used herein, a “gene disease” is a disease that may be caused by mutations in more than one gene.

As used herein, a “recessive phenotype” is a single gene phenotype whose expression is restricted to individuals who inherit a genotype with two copies of a particular gene. As used herein, a “dominant phenotype” is a single gene phenotype whose expression is restricted to individuals who inherit a genotype with at least one copy of a particular gene.

As used herein, “disease risk” refers to the likelihood that an existing person or a person born via IVF from an embryo will express a specified disease based on an interpretation of genetic data which is informed by empirical data or bioinformatic modeling.

As used herein, a “non-synonymous variant” is a DNA variant that alters the coding sequence of a gene, thereby altering the amino acid sequence of the protein product of the gene.

As used herein, “altering the gene product” (and grammatical variations thereof and the like) from a gene, refers to a change of the wild-type or normal biological function of the gene and that is caused by mutations of the gene. Alteration of the gene product from a gene includes alterations to transcription of the gene, alterations to translation of the gene, and alterations to the gene product itself.

It may be appreciated by persons of skill in the art that the discussion herein of disease, mutations, variants and other defective or negative functions are only examples of phenotypes and that such embodiments relate to any phenotype having negative, positive or neutral function.

Embodiments of the invention may provide a system and method for testing for a probability of future emergence of phenotypes in living organisms (real progeny that do not currently express the phenotypes). Living organisms may include organisms that were living at any time including those that are now dead.

In one aspect, provided herein are methods of generating a successful pregnancy probability for an embryo of a mother and a father, descriptive of the embryo's potential for a successful pregnancy if implanted; the method implemented by a computer processor executing program instructions, the method comprising the steps of: (i) providing gene sequences from a genomic DNA sample obtained from the embryo; (ii) identifying a genotype at a plurality of loci in the gene sequences; and (iii) using the processor, generating for the embryo the successful pregnancy probability, wherein the successful pregnancy probability is calculated using the genotypes identified at the plurality of loci in the gene sequences and optionally aggregating structural, karyotypal, and other factors into this probability.

In another aspect, provided herein are methods of generating a successful pregnancy probability for an embryo of a mother and a father, descriptive of the embryo's potential for a successful pregnancy if implanted; the method implemented by a computer processor executing program instructions, the method comprising the steps of: (i) providing gene sequences from a genomic DNA sample obtained from the embryo; (ii) providing gene sequences measured using a genotyping array, or sequencing, from genomic DNA samples obtained from the embryo's mother and father; (iii) using the processor, assigning a genotype based on the gene sequences present or absent in the embryo and the embryo's mother and father at a plurality of loci; and using the processor, generating for the embryo the successful pregnancy probability, wherein the successful pregnancy probability is calculated using the genotypes identified at the plurality of loci in the gene sequences and optionally aggregating structural, karyotypal, and other factors into this probability

In another aspect, provided herein are methods of treatment to reduce the risk of a woman not having a successful pregnancy with an implanted embryo and/or increasing the probability of the woman having a successful pregnancy with the embryo, the method comprising the steps of: (a) determining that the woman is at risk of an unsuccessful pregnancy with the implanted embryo based on the successful pregnancy probability generated as described herein; and (b) administering low dose heparin to the woman to increase the probability that the pregnancy will be successful and/or to reduce the risk that the pregnancy will be unsuccessful.

In another aspect, provided herein are methods of selecting an embryo for suitability for intrauterine transfer from a set of non-aneuploid embryos, the method implemented by a computer processor executing program instructions, the method comprising the steps of: (i) using the processor, generating a successful pregnancy probability as described herein for each embryo of the set of non-aneuploid embryos; (ii) including, excluding, up-prioritizing or down-prioritizing the embryo from the set of non-aneuploid embryos as suitable or unsuitable for intrauterine transfer, if the successful pregnancy probability for the embryo is better than or worse than the successful pregnancy probability for another embryo of said set of non-aneuploid embryos, or if the successful pregnancy probability falls above or below a pre-determined threshold; and (iii) repeating steps (i)-(ii) for each embryo of said set of non-aneuploid embryos.

In another aspect, provided herein are methods of ranking a set of non-aneuploid embryos for suitability for intrauterine transfer, the method implemented by a computer processor executing program instructions, the method comprising the steps of: (i) using the processor, generating a successful pregnancy probability as described herein for each embryo of the set of non-aneuploid embryos; (ii) ranking each embryo from the set of non-aneuploid embryos for suitability for intrauterine transfer based on the successful pregnancy probability of that embryo, wherein the ranking is relative to the successful pregnancy probability of another embryo of said set of non-aneuploid embryos or the ranking is relative to a pre-determined standard; and (iii) repeating steps (i)-(ii) for each embryo of said set of non-aneuploid embryos.

In another aspect, provided herein are systems for generating a successful pregnancy probability for an embryo of a mother and a father, descriptive of the embryo's potential for a successful pregnancy if implanted; the system comprising: a memory and a computer processor to: (i) receive gene sequences from a genomic DNA sample obtained from the embryo; (ii) identify a genotype at a plurality of loci in the gene sequences; and (iii) generate for the embryo the successful pregnancy probability, wherein the successful pregnancy probability is calculated using the genotypes identified at the plurality of loci in the gene sequences.

In another aspect, provided herein are systems for generating a successful pregnancy probability for an embryo of a mother and a father, descriptive in the embryo's potential for a successful pregnancy if implanted; the system comprising: a memory and a computer processor to: (i) receive gene sequences from a genomic DNA sample obtained from the embryo; (ii) receive gene sequences measured using a genotyping array from genomic DNA samples obtained from the embryo's mother and father; (iii) assign a genotype based on the gene sequences present or absent in the embryo and the embryo's mother and father at a plurality of loci; and (iv) generate for the embryo the successful pregnancy probability, wherein the successful pregnancy probability is calculated using the genotypes identified at the plurality of loci in the gene sequences.

In another aspect, provided herein are systems selecting an embryo for suitability for intrauterine transfer from a set of non-aneuploid embryos; the system comprising: a memory and a computer processor to: (i) generate a successful pregnancy probability as described herein for each embryo of the set of non-aneuploid embryos; (ii) include, exclude, up-prioritize or down-prioritize the embryo from the set of non-aneuploid embryos as suitable or unsuitable for intrauterine transfer, if the successful pregnancy probability for the embryo is better than or worse than the successful pregnancy probability for another embryo of said set of non-aneuploid embryos, or if the successful pregnancy probability falls above or below a pre-determined threshold; and (iii) repeat steps (i)-(ii) for each embryo of said set of non-aneuploid embryos.

In another aspect, provided herein are systems for ranking a set of non-aneuploid embryos for suitability for intrauterine transfer; the system comprising: a memory and a computer processor to: (i) generate a successful pregnancy probability as described herein for each embryo of the set of non-aneuploid embryos; (ii) rank each embryo from the set of non-aneuploid embryos for suitability for intrauterine transfer based on the successful pregnancy probability of that embryo, wherein the ranking is relative to the successful pregnancy probability of another embryo of said set of non-aneuploid embryos or the ranking is relative to a pre-determined standard; and (iii) repeat steps (i)-(ii) for each embryo of said set of non-aneuploid embryos.

Embodiments of the invention relate to a nucleic acid molecule comprising an annexin A5 (ANXA5) gene regulation element, which comprises at least one point mutation, whereby said point mutation (e.g., substitution) is selected from a defined subset of possible mutations.

In certain embodiments of the invention, embryos of IVF couples with a known genetic risk are screened for these particular genetic variants, using a particular technique, involving amplification of targeted regions of the human genome, using primers of a specified sequence, and using the amplicons of this process to make a particular diagnosis of affected or unaffected upon the embryo, and including this diagnosis in a report. This report serves to inform about the implantation and risk profile of the particular embryo, and the susceptibility of a pregnancy with a given embryo to treatment. Hypercoagulable disorders that promote thrombosis, collectively termed thrombophilia, are a significant genetic factor, against which the above test can screen, and for which treatment is possible for pregnancies of embryos with a given genotype. Other non-limiting examples of relevant gene variants may include variants of annexin A5 (ANXA5), chorionic gonadotropin beta polypeptide 5 (CGP5), human leukocyte antigen-A (HLA-A), human leukocyte antigen-B (HLA-B), human leukocyte antigen-C(HLA-C), human leukocyte antigen-G (HLA-G), interleukin 10 (IL-10), killer cell immunoglobulin-like receptor genes (KIR), PD1, tumor necrosis factor alpha (TNF-α), tumor necrosis factor beta (TNF-β) and/or toll-like receptor 4 (TNF4). Relevant sequence variants for the presently described method(s) may include and are not limited to protein-coding, non-protein-coding, intron sequences, exon sequences, promoter sequences, enhancer sequences, miRNA-encoding sequences, and/or other regulatory factors.

In yet another aspect, the systems and methods provided herein include error correction for an embryo genomic sequence using parent and sibling genotypes. For example, the genomes of the mother and/or father may be used for reference to detect and correct sequencing errors and improve the reliability of sequencing reads for an embryo. In some embodiments, the embryo genomic sequence is corrected using the genomes of the mother and the father, where the two genomes of the mother and father are measured using a genotyping array, while the embryo genomes are measured using a genomic sequencer. In some embodiments, the embryo genomic sequence is corrected using the genomes of the mother and the father, when the two genomes of the mother and father are measured using a genotyping array, while the embryos are measured by a genomic sequencer, or by another genotyping array. In some embodiments, each embryo genome is measured in replicate (whether measured by a genomic sequencer, a genotyping array or a combination thereof), and these replicate measurements of the genome for the same embryo are used to correct one another, into a unified, replicate-corrected embryo genome.

In combination with these replicates, further methods of error correction, using parent and sibling genotypes, may also be employed. In some embodiments, the genomic sequence of the embryo is corrected using the genomes of the mother and the father, in combination with a population reference panel, as a unified method, where the two are combined in particular by using the population reference panel as a tie-breaking vote during the process when the two parental genomes are being used to error correct, and the inferences made from the two parental genomes are inconclusive, as well as imputing with the population reference panel both prior to, and posterior to, applying error correction from the parental genomes.

An instantiation, by no means exhaustive of all possibilities covered, of the foregoing methods and systems of error connection for a genomic sequence of an embryo includes ones where the embryo and the parents have their DNA sequences initially characterized using the aforementioned technologies; and because DNA is inherited in blocks by the embryo from the original genome sequences of the mother and father, the state of a base or set of bases in a specific region in the embryo genome can be compared to the relevant regions of the maternal, paternal, and population level genomes. If, for example, all but one or a few bases in a region of the embryo genome could have originated from specific, corresponding blocks from the parents' genomes, the most likely explanation for the discrepancy is an error in reading out the embryo DNA, which has been amplified from just a few cells. Setting the discrepant base values to those on the parental or population blocks will likely correct the error. Similarly, when a read of the embryo's genome is entirely missing, setting the missing base values to those on the parental or population blocks will likely correct the error. Processes implementing correction using only parental genomes, or only population genomes, have been tested and shown to substantially improve accuracy.

In yet another aspect, method of validating that the methods and systems described herein are accurate, is to compare the genomic sequence of a cell line as measured using two different systems. One of these measurements can be treated as a baseline truth, due to having been characterized at a high level of genotyping resolution. The second of these measurements can be treated as an “embryo stand-in”, or as a test of the method or system. The second “embryo stand-in” cell line will in this case be sampled at a reduced resolution (“down-sampled”) as a way of having it play the part of an embryo DNA sample—this “embryo stand-in” low resolution is significant, because the smaller amount of DNA present in a sample from an embryo generally results in a lower resolution genome. The “embryo stand-in” genome is then fixed or corrected using the genomes of other cell lines which are parental to the “embryo stand-in”, and still other genomes which are sibling to the “embryo stand-in”, and which are, in some cases, either down-sampled in the same way, or not down-sampled—respectively representing sibling embryos, and previously birthed siblings. The methods and systems of validation can optionally be combined (or not combined) with population reference panels, as described above. Finally, the validation methods and systems described herein can also be used to validate other methods and systems described herein, such as for demonstration of the preservation of embryo ranking or embryo selection as determined using a successful pregnancy probability as described herein.

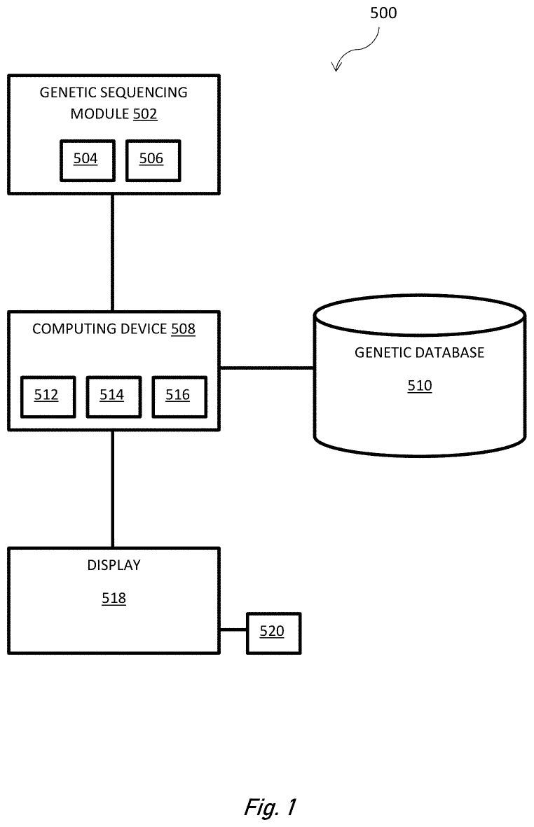

FIG. 1 is a schematic illustration of a system 500 according to an embodiment of the invention. Methods disclosed herein may be performed using the system of FIG. 1.

System 500 may include a genetic sequencing module 502 that accepts genetic material or DNA samples from an existing person or embryo and generates a genomic sequence or profile for each. Genetic sequencing module 502 may include a processor 504 for generating each genomic sequence or profile and a memory 506 for storing each genomic sequence or profile.

Computing device 508 may include, for example, any suitable processing system, computing system, computing device, processing device, computer, processor, or the like, and may be implemented using any suitable combination of hardware and/or software. Computing device 508 may include for example one or more processor(s) 512, memory 514 and software 516. Data generated by genetic sequencing module 502, such as each genomic sequence or profile, may be transferred, for example, to computing device 508. The data may be stored in the memory 514 as for example digital information and transferred to computing device 508 by uploading, copying or transmitting the digital information. Processor 504 may communicate with computing device 508 via wired or wireless command and execution signals.

Computing device 508 may use each person's or embryo's genome information to generate a genomic sequence or profile. Computing device 508 may combine the probabilities or likelihoods of a specified embryo with a specified genomic sequence or profile of being carried to term to generate a successful pregnancy probability for that embryo. Computing device 508 may repeat the process to generate a successful pregnancy probability for each of a set of embryos.

In some embodiments using a matching method, computing device 508 may compare genotypes in the embryo to genotypes in a database 510 to detect any genotype-phenotype matches. Database 510 may connect to computing device 508 via a wired or wireless connection.

In some embodiments using a scoring method, computing device 508 may compute scores and weightings associated with genotypes in the embryo or retrieve scores and weightings from an external database.

Memory 506 and 514 and database 510 may include cache memory, long term memory such as a hard drive, and/or external memory, for example, including random access memory (RAM), read only memory (ROM), dynamic RAM (DRAM), synchronous DRAM (SD-RAM), flash memory, volatile memory, non-volatile memory, cache memory, buffer, short term memory unit, long term memory unit, or other suitable memory units or storage units. Memory 506 and 514 and database 510 may store instructions (e.g., software 516) and data to execute embodiments of the foregoing methods, steps and functionality (e.g., in long term memory, such as a hard drive).

Computing device 508 may include a computing module having machine-executable instructions. The instructions may include, for example, a data processing mechanism (including, for example, embodiments of methods described herein) and a modeling mechanism. These instructions may be used to cause processor 512 using associated software 516 modules programmed with the instructions to perform the operations described. Alternatively, the operations may be performed by specific hardware that may contain hardwired logic for performing the operations, or by any combination of programmed computer components and custom hardware components.

Embodiments of the invention may include an article such as a computer or processor readable medium, or a computer or processor storage medium, such as for example a memory, a disk drive, or a USB flash memory, encoding, including or storing instructions, e.g., computer-executable instructions, which when executed by a processor or controller, carry out methods disclosed herein.

Processor 512 may perform and execute various methods described herein.

Display 518 may display results and/or intermediate data such as outcomes, probabilities, genomic profiles. Display 518 may include a monitor or screen, such as an organic light emitting diode (LED) screen, liquid crystal display (LCD) screen, thin film transistor display, or the like. In one embodiment, the user may interact with display 580 using input device(s) 520.

Input device(s) 520 may include a keyboard, pointing device (e.g., mouse, trackball, pen), a touch screen or cursor direction keys, communicating information and command selections to processor 514. Input device 520 may communicate user direction information and command selections to the processor 514. For example, a user may use input device 520 to select embryos for testing, define genes, phenotypes and/or diseases to be under investigation, set thresholds or categories, set margins of error or certainty of calculations, etc.

Processor 504 and 514 may include, for example, one or more processors, controllers, central processing units (“CPUs”), or graphical processing units (“GPUs”). Software 516 may be stored, for example, in memory 514.

Computer Systems/Processors (the following computer systems/processors may be used in combination with, or as an alternative to, computer systems/processors described in reference to FIG. 1).

The methods and systems described herein may be used in combination with one or more processors, having either single or multiple cores. The processor may be operatively connected to a memory. For instance, the memory may be solid state, flash, or nanoparticle based. The processor and/or memory may be operatively connected to a network via a network adapter. The network may be digital, analog, or a combination of the two. The processor may be operatively connected to the memory to execute computer program instructions to perform one or more steps described herein. Any computer language known to those skilled in the art may be used.

Input/output circuitry may be included to provide the capability to input data to, or output data from, the processor and/or memory. For example, input/output circuitry may include input devices, such as keyboards, mice, touch pads, trackballs, scanners, and the like, output devices, such as video adapters, monitors, printers, and the like, and input/output devices, such as, modems and the like.

The memory may store program instructions that are executed by, and data that are used and processed by, CPUs to perform various functions. The memory may include electronic memory devices, such as random-access memory (RAM), read-only memory (ROM), programmable read-only memory (PROM), electrically erasable programmable read-only memory (EEPROM), and flash memory, and electro-mechanical memory, such as magnetic disk drives, tape drives, and optical disk drives, which may be used as an integrated drive electronics (IDE) interface, or a variation or enhancement thereof, such as enhanced IDE (EIDE) or ultra direct memory access (UDMA), or a small computer system interface (SCSI) based interface, or a variation or enhancement thereof, such as fast-SCSI, wide-SCSI, fast and wide-SCSI, etc., or a fiber channel-arbitrated loop (FC-AL) interface.

The systems described herein may also include an operating system that runs on the processor, including UNIX®, OS/2®, and WINDOWS®, each of which may be configured to run many tasks at the same time, e.g., a multitasking operating systems. In one aspect, the methods are utilized with a wireless communication and/or computation device, such as a mobile phone, personal digital assistant, personal computer, and the like. Moreover, the computing system may be operable to wirelessly transmit data to wireless or wired communication devices using a data network, such as the Internet, or a local area network (LAN), wide-area network (WAN), cellular network, or other wireless networks known to those skilled in the art.

In one embodiment, a graphical user interface may be included to allow human interaction with the computing system. The graphical user interface may comprise a screen, such as an organic light emitting diode screen, liquid crystal display screen, thin film transistor display, and the like. The graphical user interface may generate a wide range of colors, or a black and white screen may be used.

In certain instances, the graphical user interface may be touch sensitive, and it may use any technology known to skilled artisans including, but not limited to, resistive, surface acoustic wave, capacitive, infrared, strain gauge, optical imaging, dispersive signal technology, acoustic pulse recognition, frustrated total internal reflection, and diffused laser imaging.

The following examples are presented to more fully illustrate preferred embodiments of the invention. They should in no way be construed, however, as limiting the broad scope of the invention.

### Examples

### Assay for IVF Miscarriage Risk: Annexin A5 M2 Haplotyping in IVF Patients and Preimplantation Embryos

### Introduction

The anticoagulant protein annexin A5 (ANXA5, also sometimes called RPRGL3) (see putative Homo sapiens ANXA5 sequence, GenBank accession no. NM_001154.4) helps maintain placental vasculature. An estimated 22% of the general population carries the M2 haplotype of the ANXA5 promoter, characterized at least by single-nucleotide polymorphisms of −19G/A, 1A/C, 27T/C and 76G/A, where nucleotide position 1 denotes the first nucleotide of exon 1. The ANXA5 M2 haplotype results in decreased protein levels affecting placental vasculature, and is associated with increased risk of miscarriage and other placental mediated pregnancy complications. Low-dose heparin treatment is an effective strategy to improve IVF success rates in M2 carrier patients from 16% to 42% clinical pregnancy rate (Fishel et al. EBioMedicine 2016). Unfortunately, at the time of the present disclosure, M2 testing is limited to cumbersome phlebotomy and sanger sequencing and is unavailable in preimplantation embryos.

This Example presents the development of a new test for evaluating M2 carrier status in IVF patient saliva and preimplantation embryos.

### Methods:

Test performance was measured by comparing Sanger sequencing on parental blood DNA and quantitative real-time (q)PCR on saliva DNA, cell line 7-cell replicates, cell line 7-cell samples and corresponding purified DNA, trophectoderm biopsies and DNA isolated from the corresponding embryonic stem cell line, Mendelian inheritance expectations in embryos, embryo Sanger sequencing, and SNP microarray-based linkage analyses.

### Results:

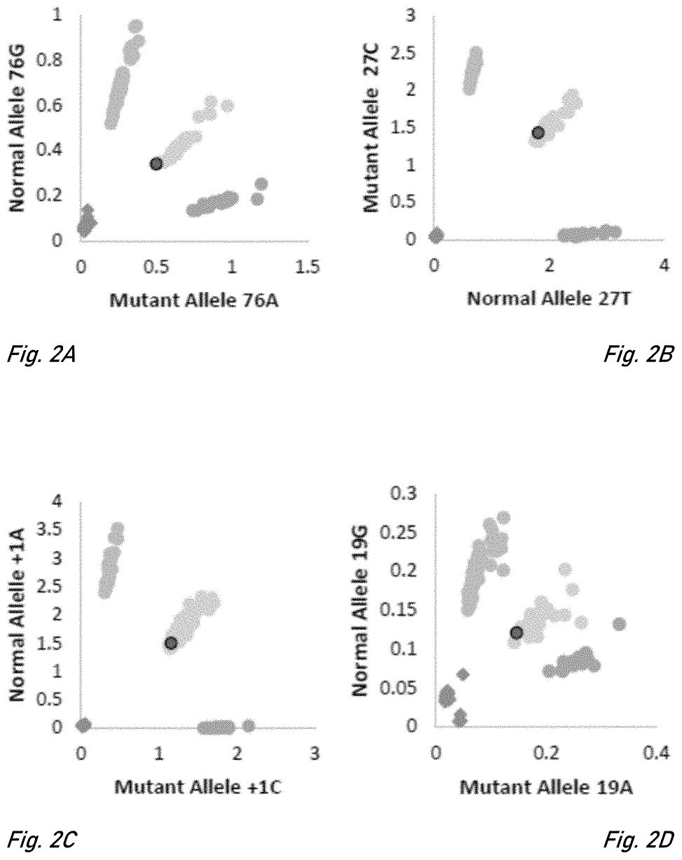

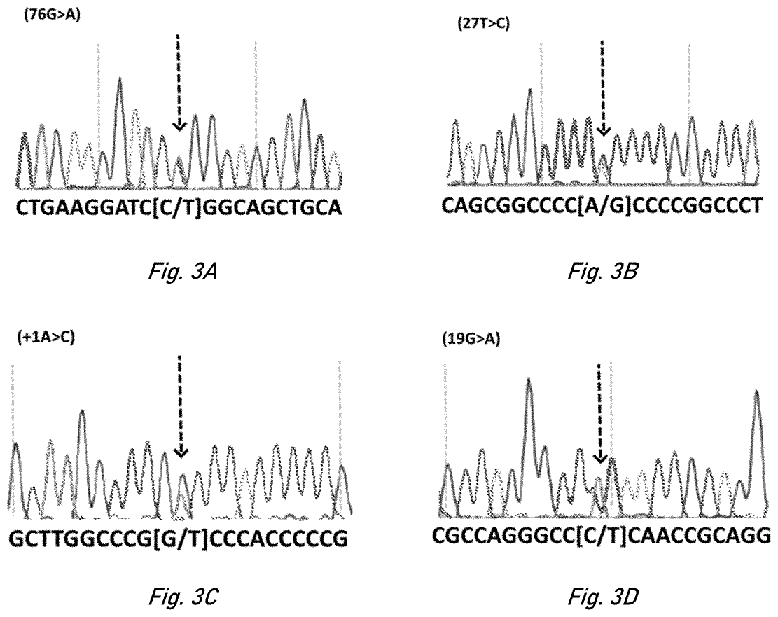

M2 saliva qPCR. 100% concordance was obtained between conventional Sanger sequencing and a novel qPCR method on saliva DNA (FIGS. 2A-2D, 3A-3D).

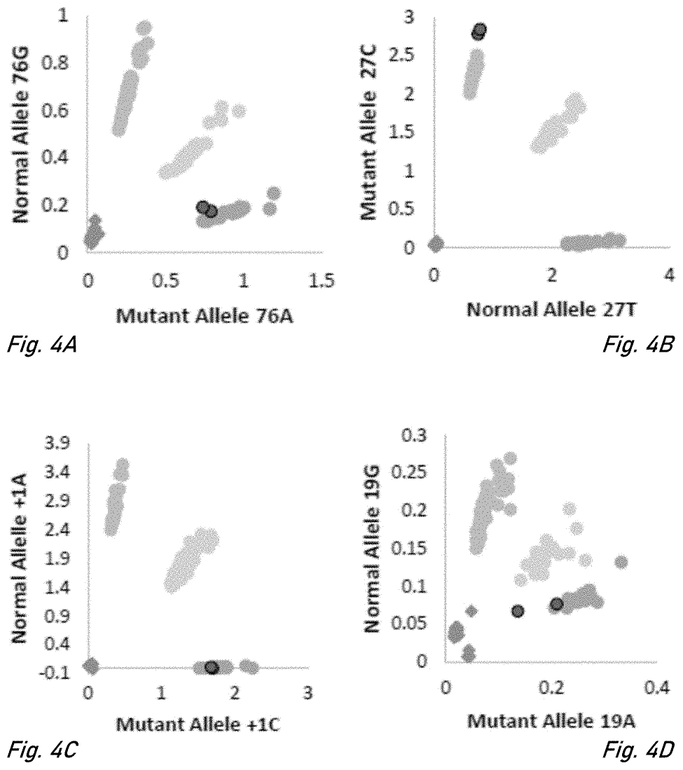

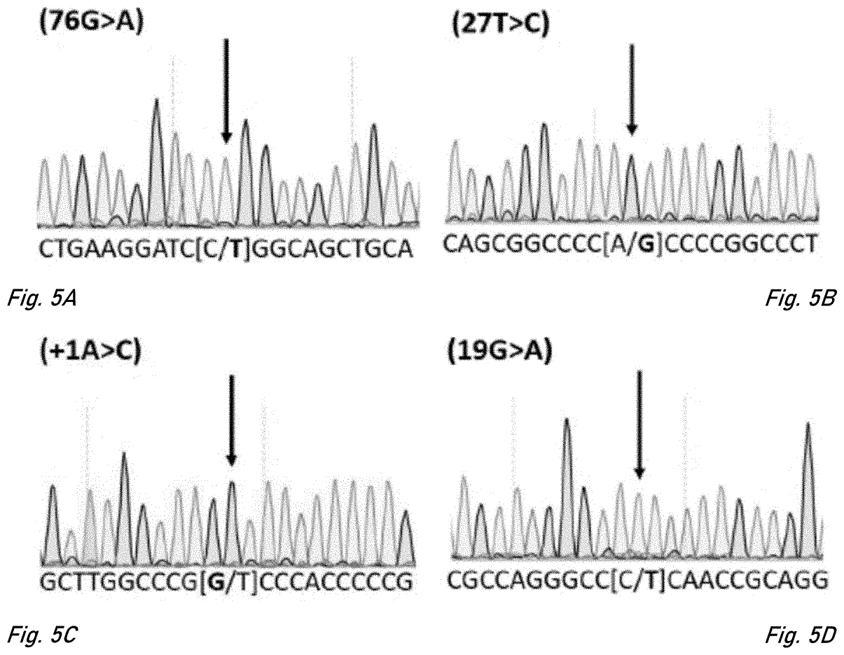

M2 PGT validation. 100% concordance was obtained between all replicates of cell line 7-cell and corresponding bulk DNA samples (FIGS. 4A-4D, 5A-5D).

M2 trophectoderm PGT. 100% concordance was obtained between qPCR and Mendelian inheritance with SNP array-based linkage analysis in human blastocyst biopsies (n=107) (FIGS. 4A-4D, 5A-5D).

### Conclusion:

A saliva DNA-based M2 haplotyping test for infertile patients, as well as a new ability to perform “PGT-M2 haplotyping” for carrier couples undergoing IVF, were developed and validated.

All references, including publications, patent applications, and patents, cited herein are hereby incorporated by reference in its entirety herein.

Having described preferred embodiments of the invention with reference to the accompanying drawings, it is to be understood that the invention is not limited to the precise embodiments, and that various changes and modifications may be affected therein by those skilled in the art without departing from the scope or spirit of the invention as defined in the appended claims.

What is claimed is:

1. A method of generating a successful pregnancy probability for an embryo of a mother and a father, descriptive of the embryo's potential for a successful pregnancy if implanted; the method implemented by a computer processor executing program instructions, the method comprising the steps of:
i. providing gene sequences from a genomic DNA sample obtained from the embryo;
ii. identifying a genotype at a plurality of loci in the gene sequences; and
iii. using the processor, generating for the embryo the successful pregnancy probability, wherein the successful pregnancy probability is calculated using the genotypes identified at the plurality of loci in the gene sequences and optionally aggregating structural, karyotypal, and other factors into this probability.

2. The method of claim 1, wherein the gene sequences comprise Annexin A5 (ANXA5) gene sequences.

3. The method of claim 2, the step of identifying the genotype at a plurality of loci in the ANXA5 gene sequences comprises identifying whether the M2 haplotype is present or absent in the ANXA5 gene sequences.

4. A method of generating a successful pregnancy probability for an embryo of a mother and a father, descriptive of the embryo's potential for a successful pregnancy if implanted; the method implemented by a computer processor executing program instructions, the method comprising the steps of:
i. providing gene sequences from a genomic DNA sample obtained from the embryo;
ii. providing gene sequences measured using a genotyping array, or sequencing, from genomic DNA samples obtained from the embryo's mother and father;
iii. using the processor, assigning a genotype based on the gene sequences present or absent in the embryo and the embryo's mother and father at a plurality of loci; and
iv. using the processor, generating for the embryo the successful pregnancy probability, wherein the successful pregnancy probability is calculated using the genotypes identified at the plurality of loci in the gene sequences and optionally aggregating structural, karyotypal, and other factors into this probability.

5. The method of claim 4, wherein the gene sequences comprise Annexin A5 (ANXA5) gene sequences.

6. The method of claim 5, the step of identifying the genotype at the plurality of loci in the ANXA5 gene sequences comprises identifying whether the M2 haplotype is present or absent in the ANXA5 gene sequences.

7. A method of treatment to reduce the risk of a woman not having a successful pregnancy with an implanted embryo and/or increasing the probability of the woman having a successful pregnancy with the embryo, the method comprising the steps of:
i. determining that the woman is at risk of an unsuccessful pregnancy with the implanted embryo based on the successful pregnancy probability generated according to the method of claim 6; and
ii. administering low dose heparin to the woman to increase the probability that the pregnancy will be successful and/or to reduce the risk that the pregnancy will be unsuccessful.

8. A method of selecting an embryo for suitability for intrauterine transfer from a set of non-aneuploid embryos, the method implemented by a computer processor executing program instructions, the method comprising the steps of:
i. using the processor, generating a successful pregnancy probability according to the method of claim 4 for each embryo of the set of non-aneuploid embryos;
ii. including, excluding, up-prioritizing or down-prioritizing the embryo from the set of non-aneuploid embryos as suitable or unsuitable for intrauterine transfer, if the successful pregnancy probability for the embryo is better than or worse than the successful pregnancy probability for another embryo of said set of non-aneuploid embryos, or if the successful pregnancy probability falls above or below a pre-determined threshold; and
iii. repeating steps (i)-(ii) for each embryo of said set of non-aneuploid embryos.

9. The method according to claim 8, wherein the set of non-aneuploid embryos are siblings.

10. A method of ranking a set of non-aneuploid embryos for suitability for intrauterine transfer, the method implemented by a computer processor executing program instructions, the method comprising the steps of:
i. using the processor, generating a successful pregnancy probability according to the method of claim 4 for each embryo of the set of non-aneuploid embryos;
ii. ranking each embryo from the set of non-aneuploid embryos for suitability for intrauterine transfer based on the successful pregnancy probability of that embryo, wherein the ranking is relative to the successful pregnancy probability of another embryo of said set of non-aneuploid embryos or the ranking is relative to a pre-determined standard; and
iii. repeating steps (i)-(ii) for each embryo of said set of non-aneuploid embryos.

11. The method according to claim 10, wherein the set of non-aneuploid embryos are siblings.

12. A system for generating a successful pregnancy probability for an embryo of a mother and a father, descriptive of the embryo's potential for a successful pregnancy if implanted; the system comprising:
a memory; and
a computer processor to:
i. receive gene sequences from a genomic DNA sample obtained from the embryo;
ii. identify a genotype at a plurality of loci in the gene sequences; and
iii. generate for the embryo the successful pregnancy probability, wherein the successful pregnancy probability is calculated using the genotypes identified at the plurality of loci in the gene sequences and optionally aggregating structural, karyotypal, and other factors into this probability.

13. The system of claim 12, wherein the gene sequences comprise Annexin A5 (ANXA5) gene sequences.

14. The system of claim 13, the step of identifying the genotype at the plurality of loci in the ANXA5 gene sequences comprises identifying whether the M2 haplotype is present or absent in the ANXA5 gene sequences.

15. A system for generating a successful pregnancy probability for an embryo of a mother and a father, descriptive in the embryo's potential for a successful pregnancy if implanted; the system comprising:
a memory; and
a computer processor to:
i. receive gene sequences from a genomic DNA sample obtained from the embryo;
ii. receive gene sequences measured using a genotyping array from genomic DNA samples obtained from the embryo's mother and father;
iii. assign a genotype based on the gene sequences present or absent in the embryo and the embryo's mother and father at a plurality of loci; and
iv. generate for the embryo the successful pregnancy probability, wherein the successful pregnancy probability is calculated using the genotypes identified at the plurality of loci in the gene sequences and optionally aggregating structural, karyotypal, and other factors into this probability.

16. The system of claim 15, wherein the gene sequences comprise Annexin A5 (ANXA5) gene sequences.

17. The system of claim 16, the step of identifying the genotype at the plurality of loci in the ANXA5 gene sequences comprises identifying whether the M2 haplotype is present or absent in the ANXA5 gene sequences.
